(19) United States
(12) Patent Application Publication
(10) Pub. No.: US 2012/0315184 A1
(43) Pub. Date: Dec. 13, 2012

(54) METHODS AND APPARATUS FOR DIFFUSE REFLECTIVE UV CAVITY AIR TREATMENT

(75) Inventor: Reginald W. Clark, San Diego, CA (US)

(73) Assignee: NOVATRON, INC., San Diego, CA (US)

(21) Appl. No.: 13/464,431

(22) Filed: May 4, 2012

Related U.S. Application Data
(60) Provisional application No. 61/483,545, filed on May 6, 2011.

(51) Int. Cl. A61L 9/20

(52) U.S. Cl. 422/4; 250/436

(57) ABSTRACT

The present disclosure relates to an apparatus and its methods of use which provides a reflective cavity technique that significantly increases the intensity and uniformity of UV energy, enabling very high and uniform UV irradiance. The high UV irradiance and high uniformity lead to previously unobtainable levels of air sterilization.

### CROSS-REFERENCE TO RELATED APPLICATIONS

This application claims priority under 35 U.S.C. §119(e) to U.S. Provisional Application No. 61/483,545, filed on May 6, 2011, the disclosure of which is hereby incorporated by reference in its entirety.

### BACKGROUND

1. Technical Field

Embodiments discussed herein relate generally to the use of ultraviolet energy to kill microorganisms and destroy contaminants in flowing air. More specifically, such embodiments relate to apparatus and methods of using high density ultraviolet light to effectively kill airborne microorganisms and destroy contaminants using a reflective cavity technique that significantly increases the intensity and uniformity of UV energy, enabling very high and uniform UV irradiance.

2. Description of the Related Technology

Ultraviolet (UV) energy has been used to kill microorganisms such as bacteria and viruses in air since the early 1900s. If sufficiently high doses of UV are applied, the technology can very effectively kill microorganisms. Low UV doses and lack of uniformity of the UV radiation can, however, significantly reduce the germicidal effects of UV. The invention described herein provides a means for multiplying the UV flux in a UV air treatment system and significantly increasing the uniformity of the UV flux, thereby creating a major improvement in capability to create high level air sterilization effects.

Unfortunately, previous UV sterilization systems were not capable of effectively and/or efficiently utilizing UV energy to destroy airborne contaminants such as microorganisms below a level wherein the safety of exposed persons was maximized. Thus, a need exists for high energy UV systems capable of having a uniform distribution necessary for the most effective process of killing dangerous and robust airborne microorganisms and destroying other contaminants.

### SUMMARY

The systems, methods and devices discussed herein each have several innovative aspects, no single one of which is solely responsible for the desirable attributes disclosed herein.

In one embodiment, an air treatment system is provided, including a chamber, the chamber including a plurality of input apertures and a plurality of output apertures, where the interior surfaces of the chamber are substantially covered by a diffuse reflective material that is greater than 95% reflective to ultraviolet (UV) light, and at least one continuous, narrow-band UV light source disposed within the chamber, where the light source is configured to emit greater than 90% of its output energy at wavelengths between 240 nm and 280 nm.

In one aspect, the at least one continuous, narrow-band UV light source can include a low-pressure mercury discharge lamp. In one aspect, at least a portion of the light source can extend through an aperture in a wall or end plate of the chamber, and a portion of the light source disposed outside of the chamber provides at least one of mechanical securement or electrical connection for the light source.

In one aspect, a ratio of the sum of the open area and interior surface area uncovered by the diffuse reflective material to the total surface area of the interior of the chamber walls can be less than 0.05. In a further aspect, the ratio of the sum of the open area and interior surface area uncovered by the diffuse reflective material to the total surface area of the interior of the chamber walls can be less than 0.01.

In one aspect, the chamber can include a input end plate including the plurality of input apertures and an output end plate including the plurality of output apertures, where a ratio of open area to the total area of each of the end plates is less than 0.4. In a further aspect, the ratio of open area to the total area of each of the end plates can be less than 0.1.

In one aspect, the air treatment system can be configured to provide an irradiance of at least 75,000 μW/cm2 at any location within the chamber. In one aspect, the air treatment system can be configured to provide an irradiance of at least 100,000 μW/cm2 at any location within the chamber. In one aspect, the air treatment system can be configured to provide an irradiance of at least 150,000 μW/cm2 at any location within the chamber.

In one aspect, the air treatment system can be configured to provide a UV dose of at least 150,000 μW-s/cm2 to air flowing through the chamber with a dwell time within the chamber of less than 2 seconds. In one aspect, the air treatment system can be configured to provide a UV dose of at least 150,000 μW-s/cm2 to air flowing through the chamber with a dwell time within the chamber of less than 1 second.

In another embodiment, an air treatment system is provided, including a chamber, the chamber including a plurality of input apertures and a plurality of output apertures, where the interior surfaces of the chamber are substantially covered by a diffuse reflective material that is greater than 95% reflective to ultraviolet (UV) light, and at least one continuous UV light source disposed within the chamber, where the air treatment system is configured to increase the irradiance at any location within the chamber to at least 10 times the irradiance of the at least one UV light source.

In one aspect, the air treatment system can be configured to provide an irradiance of at least 75,000 μW/cm2 at any location within the chamber. In one aspect, the at least one UV light source can include a narrow-band UV light source. In one aspect, at least a portion of the light source can extend through an aperture in a wall or end plate of the chamber, and a portion of the light source disposed outside of the chamber provides at least one of mechanical securement or electrical connection for the light source.

In another embodiment, an air treatment system for reducing an amount of a contaminant in air passing through the treatment system is provided, the system including a chamber, the chamber including a plurality of input apertures and a plurality of output apertures to allow passage of air containing a contaminant therethrough, where the amount of contaminant in the air can be reduced by exposure to ultraviolet (UV) light, and where the interior surfaces of the chamber are substantially covered by a diffuse reflective material that is greater than 95% reflective to UV light, and at least one UV light source disposed within the chamber, where the air treatment system is configured to increase the irradiance at any location within the chamber by a factor of at least 10.

In one aspect, the at least one UV light source can include a narrow-band UV light source. In one aspect, at least a portion of the light source can extend through an aperture in a wall or end plate of the chamber, and a portion of the light source disposed outside of the chamber provides one of mechanical securement or electrical connection for the light source. In one aspect, the air treatment system can be configured to provide an irradiance of at least 75,000 μW/cm2 at any location within the chamber. In one aspect, the air treatment system can be configured to reduce the amount of ozone in air passing through the chamber.

In another embodiment, a method of reducing the presence of a contaminant in air is provided, the method including directing air containing a contaminant into a chamber, where the amount of the contaminant can be reduced by exposure to ultraviolet (UV) light, the chamber having interior surfaces which are substantially covered by a diffuse reflective material that is greater than 95% reflective to UV light, and introducing UV light into the chamber during passage of the air containing ozone therethrough, where the UV light is reflected multiple times by the interior surfaces of the chamber to provide an irradiance of at least 75,000 μW/cm2 at any location within the chamber.

In one aspect, the contaminant can include ozone. In one aspect, introducing UV light into the chamber can include exposing the air to an irradiance of at least 75,000 μW/cm2 at any location within the chamber.

Details of one or more embodiments of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1 shows a plot of measured kill of Bacillus subtilis endospores as a function of UV dose using UV energy at wavelength of 254 nm.

FIG. 2 is a schematic illustration of a UV lamp of length L.

FIG. 3 is a plot of UV power density or irradiance at a location at a distance from the UV lamp of FIG. 2, where the distance is given as multiples of lamp length L.

FIGS. 4A and 4B schematically illustrate perpendicular and parallel airflow relative to a UV lamp such as the UV lamp of FIG. 2.

FIG. 5 is a plot of flux multiplier M within a cavity as a function of reflectivity R of the cavity walls, shown for various values of the fractional absorbing or loss area a of the cavity walls.

FIG. 6 is a perspective view of a box structure which can be used to cover mechanical support and electrical connections for a UV or other lamp.

FIG. 7A is a cross-sectional view of a UV treatment system in which portions of the lamps are disposed outside of the cavity.

FIG. 7B is another side view of the UV treatment system of FIG. 7A.

FIG. 8A shows an example of a UV treatment system having lamps configured in a fashion similar to that of FIGS. 7A and 7B.

FIG. 8B is shows an example of securement of a lamp for use in the UV treatment system of FIG. 8A.

FIG. 9 is a plot of the emission spectrum from a medium pressure mercury discharge lamp.

FIG. 10 is a plot of the emission spectrum of a pulsed flashlamp.

FIG. 11 is a plot of the emission spectrum from a low pressure mercury discharge lamp.

Like reference numbers and designations in the various drawings indicate like elements.

### DETAILED DESCRIPTION OF THE PREFERRED EMBODIMENT

Embodiments of methods and devices described herein use a reflective cavity technique that significantly increases the intensity and uniformity of ultraviolet (UV) energy, enabling very high and uniform UV irradiance. The high UV irradiance and high uniformity lead to previously unobtainable levels of air sterilization and contaminant reduction.

The reflective cavity technology described herein provides a multiplication of UV irradiance by a factor of 10 to 50 times that produced by UV lamps alone. This occurs due to cavity effects, where energy is contained and intensity increases due to accumulation of reflected energy. The reflective cavity technology described herein also incorporates the use of reflective surfaces that have a diffuse or Lambertian reflective characteristic that results in highly uniform flux throughout the cavity.

One application of the present invention is based on the fact that UV energy causes germicidal effects by disrupting the DNA of microorganisms, thereby preventing the organisms from functioning and reproducing. The most effective UV wavelengths for inactivation of microorganisms are in the 220 to 300 nm range, with peak effectiveness near 265 nm. The germicidal effects depend strongly on the amount of UV energy delivered to the organism.

UV energy density, E, is typically measured in units of microwatt-seconds/cm² (μW-s/cm²). This is a measure of the UV energy per unit area incident on the microorganism. The energy density is also referred to as the UV dose. It is the product of the instantaneous UV power density, P, and the time over which it is applied. The power density is typically measured in μWatts/cm² and is also known as UV flux or irradiance. The energy density or dose is given by:

E (μW-s/cm²)=P (μW/cm²)×t (s) (1)

The UV energy density or dose required to kill a microorganism varies significantly from one organism to another. Some organisms are much more resistant to UV than others. In particular, some organisms form endospores, which are a semi-dormant form of the organism. Endospores are quite resistant to many sterilization technologies, including heat, chemicals, x-rays, and UV.

Germicidal effects of UV are often described in terms of logs of kill. A kill level of 1 log corresponds to a reduction by a factor of 10 (one order of magnitude) of the number of viable microorganisms. For example, if 1 million microorganisms were exposed to a 1 log kill process, 100,000 would survive. Similarly, 2 logs kill corresponds to a 99% reduction, or 2 orders of magnitude reduction, and 10,000 organisms would survive from an original population of 1 million organisms. In general, kill levels of 6 logs (1 million times reduction in microorganisms) is considered to be sterilization, since at these levels of reduction, it is extremely unlikely that a sufficient number of microorganisms would survive to cause infection or illness, even if the initial population was large.

Table 1 shows a comparison of UV doses necessary for a 1 log kill of various organisms:

TABLE 1

| | | | | D-Value (90% Kill) |
|---|---|---|---|---|
| Organism | Type | Reference | Test Medium | (μW-s/cm2) |
| Bacillus subtilis (B. atrophaeus) | Bacterial spores | Novatron, 2003 | Plates, Air | 25,000 |
| Bacillis anthracis | Mixed spores | Sharp, 1939 | Plates | 4,517 |
| Influenza A | Virus | Jensen, 1964 | Air | 1,937 |
| Vaccinia | Virus | Jensen, 1964 | Air | 1,505 |
| Mycobacterium tuberculosis | Mycobacteria | David, 1973 | Air | 2,330 |
| Legionella pneumophila | G− Bacteria | Gilpin, 1984 | Water | 1,124 |
| Staphyloccus aureus | G+ Bacteria | Sharp, 1939 | Plates | 2,596 |
| Escherichia coli | G− Bacteria | Sharp, 1939 | Plates | 2,479 |

As an example of the variation in resistance between different classes of organisms, endospores of Bacillus subtilis var. niger (name recently changed to Bacillus atrophaeus) require 25,000 μW-s/cm² for 1 log kill. Two logs kill would require 50,000 μW-s/cm², etc. By comparison, Mycobacterium tuberculosis (TB), which is a vegetative (fully metabolizing) organism, requires only 2330 μW-s/cm² for 1 log kill. Thus, the UV energy density required for 1 log kill of B. subtilis endospores should produce more than 10 logs kill of the TB bacteria.

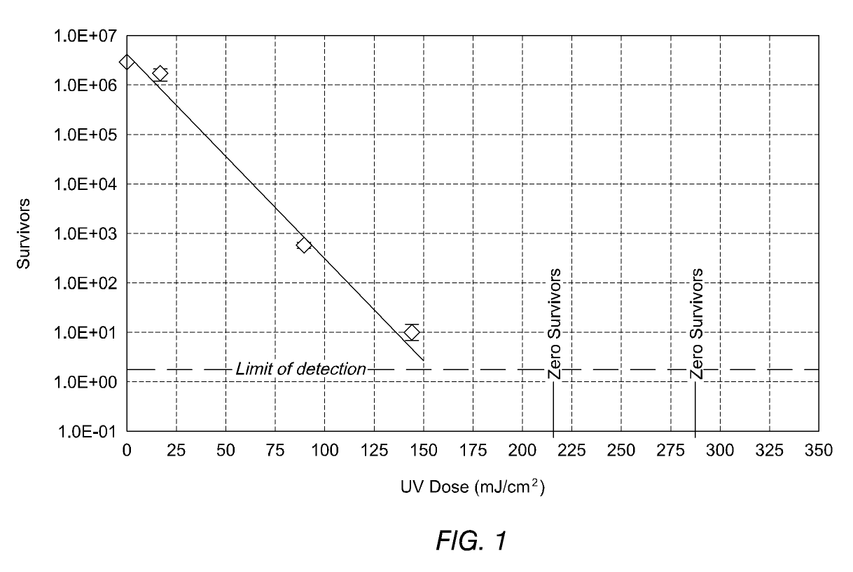

FIG. 1 is a plot of measured kill data for Bacillus subtilis endospores as a function of UV energy density (also known as UV dose) at a wavelength of 254 nm. The data show a dose response (dose requirement) of approximately 25,000 μW-s/cm² per log of kill. The dose required for 6 logs kill (considered full sterilization) is 150,000 μW-s/cm².

Using the reflective cavity technology irradiances in excess of 150,000 μW/cm² can be produced. This means that the 150,000 μW-s/cm² dose required for 6 logs kill of UV resistant endospores such as Bacillus subtilis can be delivered with residence times of 1 second or less, enabling single pass sterilization of air flowing at speeds of several hundred to more than 1000 ft/min.

While some organisms can be killed at lower levels than others, application of sufficient UV energy or dose to kill the most resistant organisms is necessary to assure that all organisms are killed. Since the UV dose is the product of the instantaneous UV irradiance and the time over which it is applied, both factors are important in achieving high kill levels. Applying high UV irradiance for sufficient long times to produce high doses for air travelling at high speeds in an air duct is difficult. The UV flux multiplication technology described herein provides a means for accomplishing this.

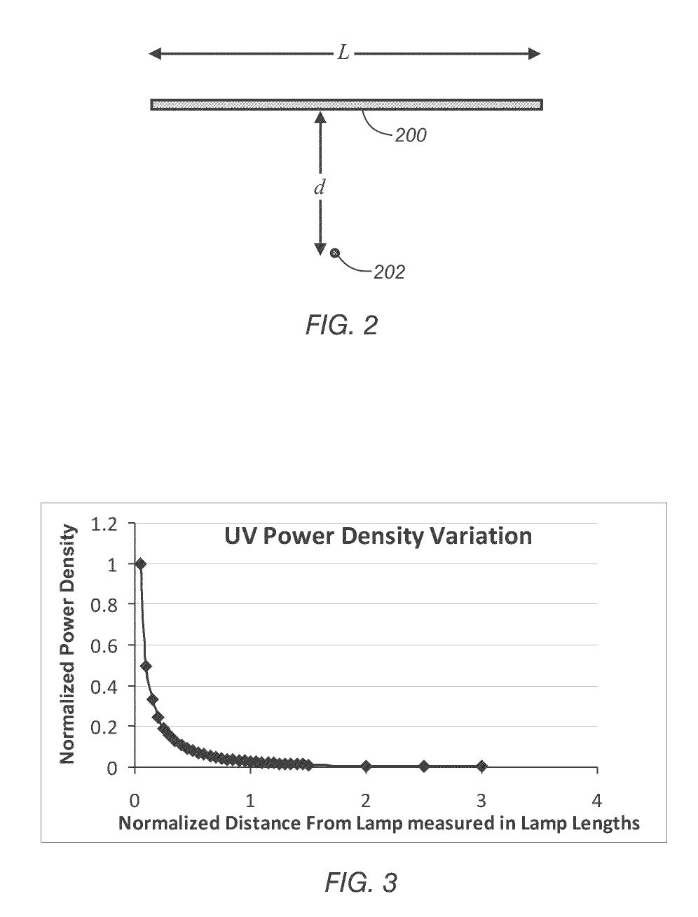

The irradiance from a UV lamp depends strongly on the distance from the lamp, decreasing rapidly as distance from the lamp increases. FIG. 2 shows a lamp 200 of length L, positioned in an air stream. In particular, FIG. 2 illustrates a geometry for calculation of UV irradiance at a point 202 located a distance d from a lamp of length L. The UV irradiance, P, measured at point 202 located a given distance d from the lamp can be calculated if the total lamp UV output power and lamp length L are known.

FIG. 3 shows a plot of the normalized power density, also known as irradiance, at the lamp axial mid-point as a function of normalized distance d from the lamp. In the plot, the distance d from the lamp is normalized as a fraction of the lamp length, L, and the UV power density is normalized to the power density at d=0.05 L. As can be seen from FIG. 3, the UV irradiance decreases very rapidly with distance from the lamp. For example, at a distance of d=0.5 L from the lamp, the UV irradiance is only about 8% of the irradiance at d=0.05 L. This creates non uniformity and regions in the air stream where the irradiance is low. If even a small fraction of the air stream is not treated effectively, the microbial kill level achievable is significantly reduced.

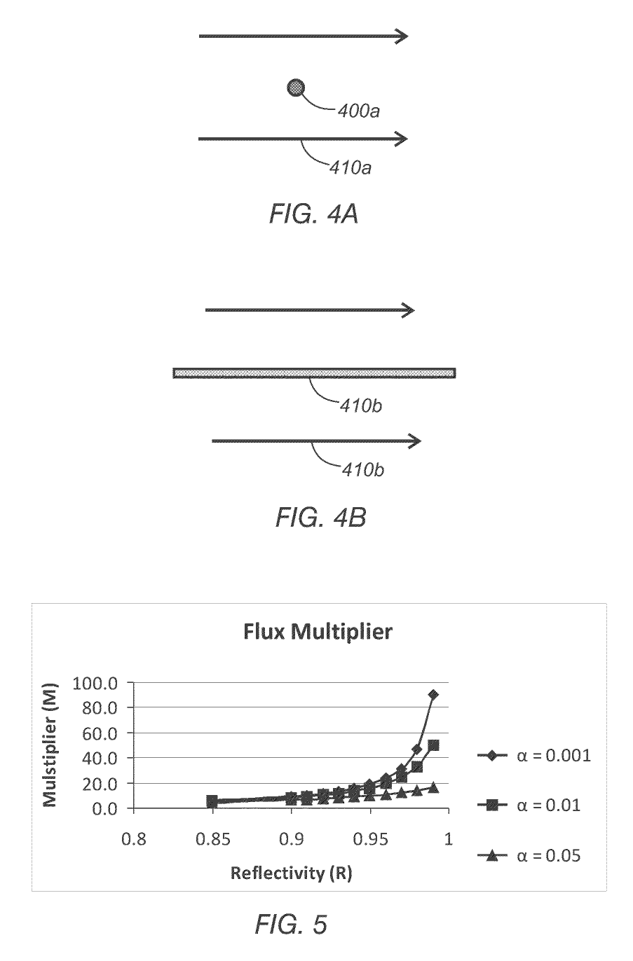

FIG. 4A is a schematic drawing showing the geometry for a cylindrical UV lamp 400a arranged with its cylindrical axis perpendicular to an airflow stream schematically illustrated as 410a. Although illustrated as discrete arrows, the lamp 400a is located within the airflow stream 410a, such that some air flows close to the lamp 400a and some air remains distant from the lamp 400a. In this arrangement, even the air which passes close to the lamp 400a spends only a very short time near the lamp 400a where the irradiance is reasonably high. As a result, the UV dose, which is the product of the irradiance and the exposure time, is low because of the short exposure time. The low UV dose results in low microbial kill.

FIG. 4B is a schematic drawing showing the geometry for a cylindrical UV lamp 400b arranged with its cylindrical axis parallel to an airflow stream schematically illustrated as 410b. In this arrangement, as in the arrangement of FIG. 4A a large portion of the air stream is distant from the lamp 400b at locations where the irradiance is low. Even though the exposure time is increased due to the parallel positioning of the lamp 400b, the irradiance is still low because of the low irradiance of the portion of the air stream distant from the lamp, resulting in low microbial kill.

To illustrate the issue, a typical germicidal lamp 71 cm (28 inches) long producing, a total UV power of 13.5 watts, would produce an irradiance of about 8500 μW/cm² at a distance d=0.05 L=3.6 cm (1.4 inches) from the lamp. At a distance d=0.5 L=36 cm (14 inches), the irradiance would be only about 700 μW/cm². If the air is travelling at a velocity of 500 ft/min=254 cm/sec, which is a typical air velocity for air sterilization applications, the air in FIG. 4A would spend only a very short time near the lamp and would reach the point d=36 cm in only about 0.14 seconds. The total accumulated dose, which is determined by the product of the irradiance at each point along the trajectory and the exposure time at that irradiance, would be very low (less than about 1,000 μW-s/cm²). Thus, resistant organisms such as Bacillus subtilis, which as discussed earlier, require about 25,000 μW-s/cm² for one log kill would not be efficiently killed with such an arrangement.

The invention described herein provides a means of achieving highly uniform UV doses in excess of 150,000 μW-s/cm² in air, resulting in more than 6 logs kill of UV resistant organisms such as B. subtilis endospores. Achieving such high doses with high uniformity is not feasible with conventional UV air treatment techniques. However, embodiments of the invention described herein use a reflective cavity technique that significantly increases the intensity and uniformity of UV energy, enabling very high and uniform UV irradiance. The high UV irradiance and high uniformity lead to previously unobtainable levels of air sterilization.

Conventional approaches to treating air with UV typically consist of inserting a lamp or array of lamps into an air duct, either with or without specular reflective material in the vicinity of the lamps. These “open duct” techniques do not produce a substantial increase in UV irradiance and the uniformity of the irradiance is poor. As a result of the low UV irradiance and significant variation in irradiance with position in the air stream, germicidal effects are limited.

Conventional UV systems often use specular reflectors to concentrate UV energy. Such reflectors are typically constructed of polished aluminum or chemically polished and anodized aluminum materials sold under trade names such as Alzak, Alanod, Miro, etc. These materials have specular reflective properties and typically have reflectance at UV wavelengths below 300 nm in the 80% to 90% range with some having total reflectance at visible wavelengths as high as 95%. Such specular reflective materials located near a lamp or multiple lamps in an open duct provide some local focusing and concentration of UV flux, increasing in the UV flux locally, but decreasing it in other locations. They do not provide the uniform, very high flux achievable with the diffuse, high reflectivity cavity technology described herein.

The reflective cavity technology described herein provides a multiplication of UV irradiance by a factor of 10 to 50 times that produced by UV lamps alone. This occurs due to cavity effects, where energy is contained and intensity increases due to accumulation of reflected energy. The reflective cavity technology described herein also incorporates the use of reflective surfaces that have a diffuse or Lambertian reflective characteristic that results in highly uniform flux throughout the cavity.

Irradiances in excess of 75,000 μW/cm², in excess of 100,000 μW/cm², and in excess of 150,000 μW/cm² can be produced using the reflective cavity technology. Because of the uniformity that can be achieved, these irradiances may be minimum irradiance levels anywhere within a treatment chamber. This means that for an irradiance in excess of 150,000 μW/cm², the 150,000 μW-s/cm² dose required for 6 logs kill of UV resistant endospores such as Bacillus subtilis can be delivered with residence times of 1 second or less, enabling single pass sterilization of air flowing at speeds of several hundred to more than 1000 ft/min. Similarly, for an irradiance in excess of 75,000 μW/cm², the necessary dose of 150,000 μW-s/cm² can be delivered with a residence time of 2 seconds. Without the reflective technology 10 to 50 times as many lamps would be required to achieve these sterilization effects and the uniformity would be poor. Such a large number of lamps is not only undesirable in terms of the electrical energy that would be consumed, it is not feasible in terms of cost and the amount of physical space that would be required to install such a large number of lamps.

Uniformity is an important factor in microorganism kill, since regions where air is exposed to lower levels of UV can significantly degrade the overall kill achieved. As described above, the irradiance from a UV lamp depends strongly on distance from the lamp. As a result, in conventional UV systems, the UV irradiance varies significantly from one location in the treatment region to another. If only a few percent of the air flowing through the treatment region is under-treated, the over-all kill level will be significantly decreased. For example, if 10% of the air experiences negligible germicidal effects, the maximum kill level that can be achieved is only about 1 log.

The reflective cavity technology described herein provides a solution to this problem by producing a very high level of uniformity throughout the cavity. This occurs because the cavity uses diffuse reflective surfaces to assure that UV energy reflects to every location in the cavity from every direction. The large number of reflections from all directions within the cavity add together to create a very uniform UV irradiance. UV irradiance has been measured in such cavities to be uniform within a few percent throughout the cavity, including in the center, in the corners and at the edges of the cavity. This is a unique capability of the reflective cavity technology that is not obtainable from other approaches and that is essential for achieving high microbial kill levels.

Since the absorption lengths for UV in air are long (on the order of kilometers), the UV photons, if contained in a highly reflective cavity, can make many reflections before they are lost by absorption or escape through an opening. It is possible to significantly increase the flux density or irradiance in such a cavity over that which would be possible in the absence of the reflecting cavity.

The reflective cavity technology described herein involves the use of a highly reflective material having diffuse reflective properties. This material is used to line the walls of a region through which air flows, creating a highly reflective diffuse reflective cavity. The diffuse reflective material has a Lambertian reflective property as opposed to a specular reflective property. This is an important feature. For surfaces with a Lambertian reflective characteristic, UV energy incident on the surface is reflected over a broad range of angles. Surfaces that exhibit specular reflective properties reflect UV rays at an angle to the surface that is the same as that of the incident rays. This does not provide the degree of flux uniformity achievable with a Lambertian reflection characteristic.

A diffuse reflective air treatment cavity, if designed properly, is analogous to an integrating sphere, which is typically a hollow sphere with at least one small opening through which light enters. In the case of the air treatment reflective cavity, provisions must be made, of course, for entry and exit of the air into and out of the cavity. This leads to some loss of light from the cavity, but it is still possible to obtain significant increases in UV flux density within the cavity.

Integrating sphere optics equations can be used to approximate the photon flux within a highly reflective air treatment cavity. While derived for a spherical geometry, the results are based on an infinite power series of multiple reflections and give a reasonable approximation of a non-spherical geometry as long as the overall dimensions (length, width, etc.) are approximately equal.

The irradiance on the inside surface of an integrating sphere is given by the equation:

$${P}_{s} = \frac{{P}_{i n}}{{A}_{s}} x \frac{R}{1 - R ( 1 - α )} = {P}_{i n} \frac{M}{{A}_{s}} , \qquad ( 2 )$$

where Pₛ is the irradiance or flux density in W/cm² near the inner surface of the sphere, R is the reflectivity of the walls, Aₛ is the total internal surface area and α is the fractional open or absorbing area of the surface. The “multiplier”, M, is a figure of merit given by:

$$M = \frac{R}{1 - R ( 1 - α )} . \qquad ( 3 )$$

This term represents an increase in irradiance due to multiple reflections. For example, the multiplier can be as large as 50 when R=0.99 and the value of α is about 0.01. Values for a for properly designed diffuse reflective cavity air treatment systems generally fall in the range of 0.01-0.05. Thus, the ratio of a sum of the open areas and interior surface area uncovered by the diffuse reflective material to the total surface area of the interior surfaces of the chamber may be between 0.01-0.05, as area uncovered by the diffuse reflective material may function similar to absorbing areas in terms of the effect on the multiplier M. Thus the UV irradiance can be increased by a factor as large as 50 in practical systems. The dependence of the multiplier on reflectivity of the chamber walls and α, as calculated from equation 3, is shown in FIG. 5.

As can be seen from FIG. 5, the irradiance or flux density multiplication factor, M, depends strongly on reflectivity of the cavity walls. The material sold by W.L. Gore under the trade name DRP has reflectivity in the UV greater than 99%. The material also has diffuse or Lambertian reflective characteristics as opposed to specular reflective characteristics. This diffuse reflective property assures a highly uniform distribution of flux density within the cavity.

An engineering model based on Equations 2 and 3 has been developed and tested and used in the design of reflective air treatment cavities to accurately predict their performance. Losses such as those due to non-reflecting or low reflectivity areas (entrance and exit areas for air and other low reflectivity or non-reflecting areas) are accounted for by means of the factor α in equation 3.

From equations 2 and 3 and FIG. 5, it can be seen that in order to avoid a significant decrease in the value of the flux multiplier, M, and a resulting degradation of the cavity performance, it is necessary to carefully control the fractional loss or absorbing area, α. The parameter α is determined by the fraction of open area at the entrance and exit to the cavity required for air flow into and out of the cavity and by the surface area of any areas located in the cavity that are not covered with highly reflective diffuse reflective material.

Plates with holes, slots or other openings can be used at each end of the cavity to allow for the entrance and exit of air while containing the UV energy. The fractional open area of these plates affect the value of the flux multiplication factor, M and the amount of pressure drop created by airflow through the cavity. The fractional open area must be selected to contain enough of the UV flux to create a significant value of the multiplier, M without creating an excessive airflow pressure drop. The airflow pressure drop associated with a perforated plate depends strongly on the fraction of open area of the plate and the velocity of the airflow. The pressure drop also depends to some extent on the shape and size of the openings, but the overall fraction of open area and the airflow velocity are more dominant parameters. Fractional ratios of open area to total plate area in the range of 0.1 to 0.4 have been generally found to be useful for most of the reflective cavity systems developed to date.

Another factor that can significantly affect the performance of the cavity is the presence of objects such as electrical connectors for the lamps, wires, ceramic end pieces on the lamps, mechanical clamps for supporting lamps, etc. Accordingly, in order to maintain a high value of the multiplier, M, and thereby improve and optimize the performance of the cavity, innovative means are desirable for supporting the lamps and providing electrical power to the lamps without introducing UV absorbing areas and materials into the cavity. If such means are not employed, the performance of the cavity and the level of UV irradiance that can be achieved will be significantly reduced.

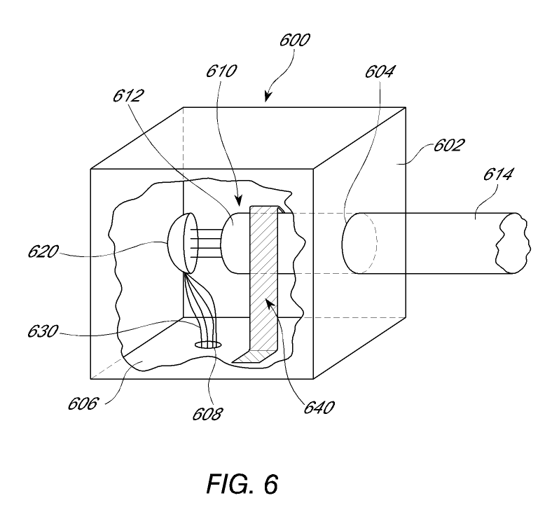

One means for minimizing UV absorbing areas in the cavity is to cover the electrical connectors and mechanical supports and mounts for the lamps with structures that are in turn covered or coated with a highly reflective diffuse reflecting material. FIG. 6 shows a perspective view of a box-like structure which encases the end portion of a lamp and the associated mechanical and electrical connections to support and provide power to the lamp.

The exposed exterior surfaces of shielding structure 600 of FIG. 6 are covered with a diffuse reflective material such as expanded PTFE. An aperture 604 formed in one of the walls 602 of shielding structure 600 allows at least the bulb portion 614 of lamp 610 to extend therethrough, while at least part of the end portion 612 of the lamp 610 is encased within the shielding structure 600. Because the end portion 612 of the lamp 610 may include a ceramic cap or similar structure which is not highly reflective, encapsulation within the shielding structure 600 will maintain a high value of the multiplier M. In addition to encapsulating the end portion 612 of lamp 610, mounting structures 640 and electrical connectors 620 and the associated wiring 630 may be encapsulated within the shielding structure 600, as well as apertures 608 formed in the side 606 of a UV treatment chamber to allow the wiring 630 to enter the chamber.

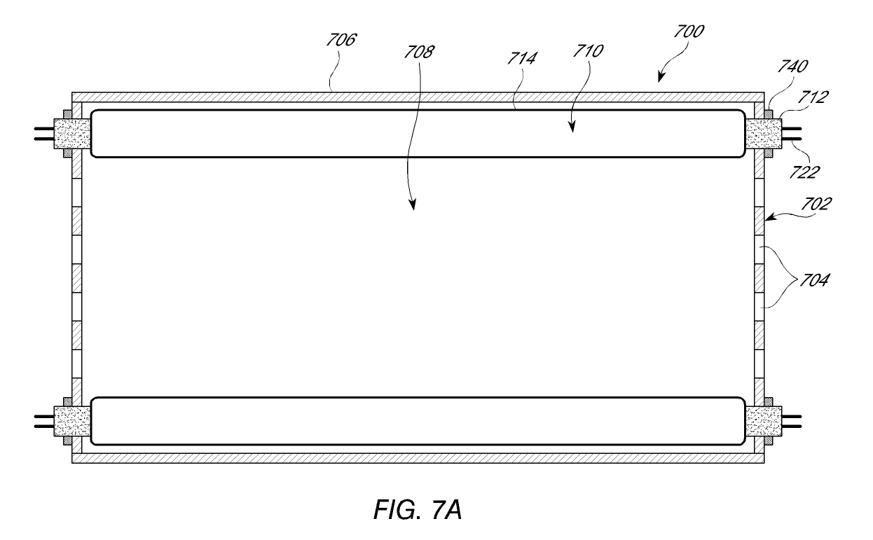

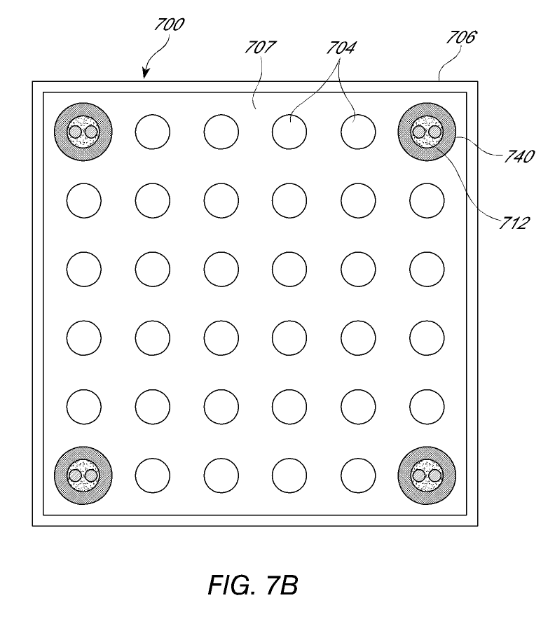

FIGS. 7A and 7B illustrate an alternative mounting method for lamps in order to maximize the reflective surface area within a UV treatment system. As can be seen in FIG. 7A, the treatment system 700 includes a chamber 708 defined by sidewalls 706 and perforated end panels 702 having apertures 704 formed therein. Some of these apertures 704 may be dimensioned to receive at least a portion of UV light sources such as UV lamps 710 therein so that at least a portion of the lamps 710 extend through the aperture. In the illustrated embodiment, the end caps 712 of the lamps are partially disposed within the apertures 704, although in other embodiments, the end caps 712 may be located fully outside chamber 708, such that only the bulbs 714 of lamps 710 are located within the chamber 708. Connectors 722 may be used to connect the lamps 710 to a power source (not shown). A wire clamp or other securement device 740 may be used to retain the lamps 710 in place. Thus, the portion of the UV lamp 710 disposed outside of the chamber can provide at least one of electrical connection and mechanical securement without the need to encase the structures providing the electrical connection or mechanical securement within a diffuse reflective material.

In the illustrated embodiment, the lamps 710 extend between opposing perforated end panels 702, although in other embodiments, the lamps 710 may extend between sidewalls 706, or may extend from only a single wall or panel in a cantilevered structure

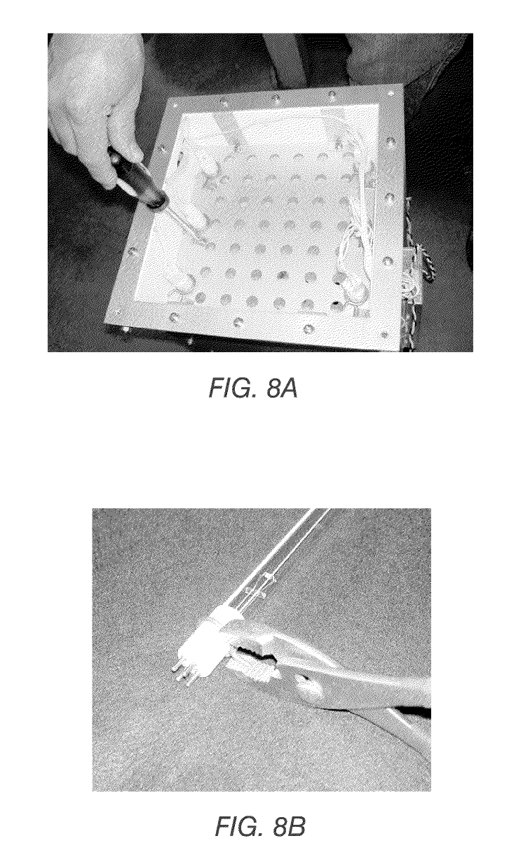

FIG. 8A shows an example of a UV treatment system in which UV lamps are supported by a perforated plate using a half clamp holder and wire ring clamp, in a manner similar to the arrangement schematically depicted in FIGS. 7A and 7B. As discussed above, this arrangement provides an effective, low cost means of supporting the UV lamps in a manner that reduces absorption in a diffuse reflective cavity by minimizing the loss factor α, allowing higher flux multiplication to be achieved.

FIG. 8B is a picture showing the attachment of a wire ring clamp to a UV lamp as a means of securing and supporting the lamp in a diffuse reflecting cavity while minimizing the loss factor, α, for the cavity and maximizing flux multiplication, as discussed above with respect to FIGS. 7A, 7B, and 8A.

In order to achieve a high value of the multiplier, M, in equation 3 and thereby realize high values of flux, it is also important to minimize the parameter α by minimizing losses of UV energy through openings in the cavity. However, openings are necessary so that air can enter and exit the cavity. Minimizing the open area for air entry and exit minimizes the loss parameter α, but increases the air pressure drop required for a given airflow through the duct/cavity. Therefore, a compromise is required to achieve a desired value of flux in the cavity with a pressure drop for the desired airflow that is tolerable.

The UV dose delivered to a volume of air passing through a cavity depends on the UV flux in the cavity and the exposure time as described in Equation 1 above. For air flowing through a cavity, the exposure time is the time required for a volume of air to transit through the cavity. The transit time is determined by the velocity of the air stream and the length of the cavity. The velocity is determined by the volumetric airflow rate and the cross-sectional area of the cavity. These quantities are related by the following equations:

v=Q/Aₓ (4)

t=L/v=LAₓ/Q, (5)

where v is airflow velocity, Q is the volumetric airflow, L is the length of the cavity and Aₓ is the cross-sectional area of the duct/cavity perpendicular to the direction of airflow. Low velocity is desirable both to maximize exposure time and to minimize airflow pressure drop. For a given volumetric airflow requirement, Q, the velocity can be reduced by increasing the cross-sectional area, Aₓ but this increases the surface area of the cavity, which can be seen from equation 2 to cause a decrease in the UV flux, or, alternatively, a requirement for additional UV input power to maintain the desired flux level. Thus, all of these parameters must be balanced to achieve an optimum cavity design. In general, cavity system designs with airflow velocities in the neighborhood of a few hundred feet per minute to about 1500 feet per minute and perforated plate open fractional areas of about 0.1 to 0.4 have been found to result in good performance for air sterilization systems. For high airflow velocities, it is usually necessary to use a perforated plate with a higher fraction of open area in order to avoid excessive airflow pressure drops. A higher fraction of open area required for high velocity, combined with the fact that the high velocity reduces exposure time, generally makes it desirable to increase the length, L, of the cavity. However, increasing the length to increase exposure time or increasing the cross-sectional area to reduce the air velocity increases the size of the cavity, which can significantly increase the cost of the air sterilization system.

Achieving an optimum design for a given air sterilization application requires determining: (1) the level of dose required to achieve the desired kill level for the organism to be destroyed, (2) the volumetric airflow requirement, (3) the acceptable pressure drop and (4) any system size or shape restrictions that may exist. Based on this information the length, cross-sectional area, perforated plate design and input UV power required can be chosen to meet desired performance criteria.

The type of lamp used to provide the UV flux is another important factor in the performance of diffuse reflective cavity air sterilization devices. A number of different types of UV lamps are available commercially. Examples of commercially available UV lamps include: medium pressure mercury discharge lamps, pulsed flashlamps, and low pressure mercury discharge lamps. Medium pressure mercury discharge lamps and microwave excited medium pressure UV lamps operate at a higher pressure than low pressure mercury lamps and produce a broader spectrum of electromagnetic energy than low pressure mercury lamps.

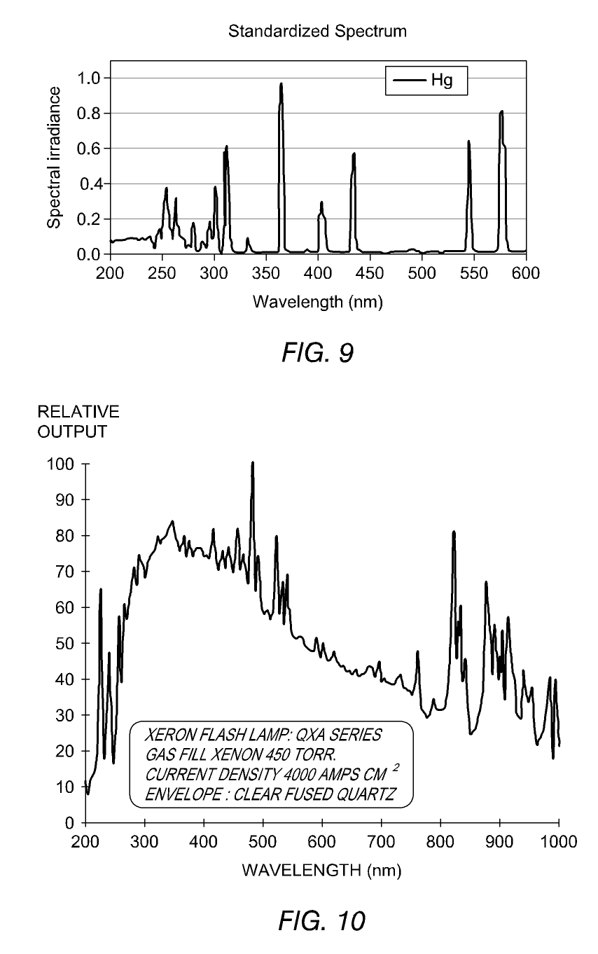

FIG. 9 is a plot showing the measured emission spectrum from a standard medium pressure mercury discharge lamp, generated by Heraeus-Noblelight GmbH. The plot shows that the emitted spectrum from a medium pressure mercury lamp is broad, and the spectrum from a medium pressure lamp extends from below 200 nm into the visible. Much of the emitted energy is wasted or not useful since it is not near the optimum wavelength for antimicrobial effects and is not effective for killing microorganisms. As noted above, the optimal wavelength for such treatment is roughly 265 nm, and thus it is advantageous if a large proportion of the output energy is between 240 and 280 nm. It is especially advantageous if 90% or more of the output energy is between 240 and 280 nm.

FIG. 10 is a plot of a measured emission spectrum from a pulsed flashlamp, generated by Q-Arc, Ltd. The plot shows that the emitted spectrum from a pulsed flashlamp is broad, and pulsed flash lamps emit a broad spectrum of wavelengths which include significant energy in the visible portion of the spectrum. Much of the emitted energy is wasted or not useful since it is not near the optimum wavelength for antimicrobial effects and is not effective for killing microorganisms. Both medium pressure mercury lamps and pulsed flash lamps can produce high irradiance, but much of the power is emitted at wavelengths that are not useful, since they are not effective for microbial kill and therefore represent wasted power and energy.

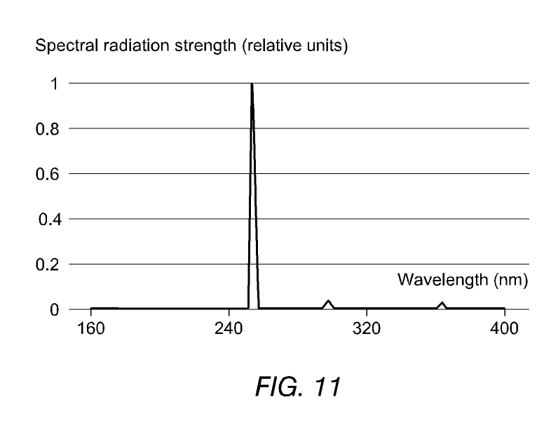

FIG. 11 is a plot of a measured emission spectrum from a standard low pressure mercury discharge lamp, generated by Heraeus-Noblelight GmbH. Low pressure mercury discharge lamps, frequently referred to as germicidal UV lamps, emit strong mercury atomic line radiation in a narrow line centered at 253.7 nm. This strong mercury emission line can contain approximately 90% of the emitted energy. The wavelength of the 253.7 nm line is very close to the optimum wavelength for germicidal effectiveness, making low-pressure mercury lamps an efficient source of UV for germicidal applications, which is approximately 265 nm. In addition, the power supplies and lamps for low pressure mercury systems are considerably less costly than those for medium pressure mercury and pulsed flash lamp systems. Thus, the use of continuous lamps may be advantageous both because of the output spectrum of available continuous lamps, such as low pressure mercury systems, as well as the relative cost and ease of use in comparison to pulsed lamps. In addition, when the timing between pulses approaches the dwell time of air passing through a treatment system, variance in doses received by particles passing through the chamber at different times increases, and the irradiance of a pulsed lamp must be increased to ensure that the minimum received dose is sufficient for the desired level of treatment. Thus, the efficiency associated with concentration of the emitted UV energy at a wavelength very near the optimum wavelength for microbial kill and their lower cost make low pressure mercury lamps an excellent source of UV for germicidal applications.

In the past, a limiting feature of low pressure mercury discharge lamps has been that the irradiances produced by these lamps is not high enough to provide high levels of air sterilization in rapidly flowing air. However, it has been demonstrated that by combining these lamps with the reflective cavity technology described herein, sufficiently high levels of irradiance can be produced to provide high levels (>6 logs) of single pass air sterilization even for highly UV resistant microorganisms such as Bacillus subtilis in air passing through the cavity in times of approximately 1 second. Low pressure mercury lamp diffuse reflective cavity systems have been built and tested that provide measured irradiance greater than 200,000 μW/cm² in cavities sized for up to 60,000 cfm with air transit times the order of 1 second.

An example of a practical diffuse reflective cavity air treatment system of a convenient size that has been constructed and tested is given below:

Example - Diffuse Reflective Cavity Air Treatment System

| | |
|---|---|
| Nominal Airflow cfm) | 3500 |
| Nominal Airflow Range (cfm) | 2000-7000 |
| Internal Dimensions (inches) | |
| Width | 24 |
| Height | 48 |
| Length | 72 |
| Pressure Drop at Nominal Airflow (iwg) | 0.8 |
| Electrical Power (Watts) | 3200 |
| UV Irradiance (μW/cm²) | 188,000 |
| UV Dose at Nominal Airflow (μW-s/cm²) | 154,000 |

In addition to the use of intense UV to sterilize air by killing airborne microorganisms, the reflective cavity technology described herein can be used to accomplish other air treatment effects such as destruction of contaminants such as ozone and other chemical substances in air streams.

For example, ultraviolet energy at the proper wavelength interacts with ozone to disassociate it into atomic and molecular oxygen. The wavelength used to kill microorganisms, approximately 254 nm, is also effective for disassociation of ozone. The differential equation for the interaction rate between UV photons and the ozone molecules is:

dN(t)=N(t)σφdt, (6)

where N(t) is the number density of ozone molecules as a function of time, σ is the cross section for the interaction, φ is the photon density and t is time. This differential equation has the solution:

N(t)=N₀exp(−σφt), (7)

where N₀ is the initial ozone concentration.

This equation can be solved to give the product of photon density and time required for a desired level of ozone destruction:

φt=−ln(N_{F}/N₀)/σ, (8)

where N_{F} is the final density of ozone remaining at the end of the destruction process.

For UV at a wavelength of 254 nm, references in the scientific literature give a value for σ of σ=1.4×10⁻¹⁷.

The photon density, φ, is given by:

φ=Pᵤᵥ/hν, (9)

where Pᵤᵥ is the UV irradiance (Watts/cm²), h is Planck's constant and ν is the frequency associated with the UV wavelength. For a wavelength of 254 nm, hν=7.8×10⁻¹⁹ Joules/photon.

For a desired level of ozone reduction, equation 8 and equation 4 give the UV energy density or dose, E, which is the product of UV irradiance and time as:

E=Pᵤᵥ×t=−(hν/σ)×ln(N_{F}/N₀)=−0.0557 ln(N_{F}/N₀). (10)

As an example, for an initial ozone level of 40 ppm and a final ozone level of 2 ppm, equation 10 gives a required dose of:

E=−0.0557×ln(2/40)=166,862 μW-s/cm². (11)

This dose that can be delivered to air flowing at high speed through a diffuse reflective cavity system in a time the order of 1 second. This would not be practical in a conventional UV air treatment system that does not employ the diffuse reflective cavity technology described herein.

In the above example, the reduction of ozone content from 40 ppm to 2 ppm represents a reduction of [(N_{F}−N₀)/N₀]×100=95%. As an additional example, for a reduction of 99%, equation 10 predicts that a dose 256,507 μW-s/cm² would be required. Such a dose and ozone reduction level could be achieved in a diffuse reflective cavity UV system with an air transit time of less than 1.5 seconds. Such a dose and ozone reduction level is practical and realizable using the diffuse reflective cavity technology, but is not practical with a conventional open UV system.

The above analysis and the calculations therein provide a reasonable approximation of contaminant reduction level such as ozone when the concentration of the contaminant in air is small. However, the above calculations do not account for the decrease in UV irradiance in the diffuse reflective cavity that can occur due to the absorption of UV photons by contaminant such as ozone. When the process is applied to high concentrations of the contaminant, the amount of reduction in contaminant content will be decreased due to increased absorption of the UV light by the denser contaminant. However, it is understood that, in general, it is possible to compensate for such absorption effects by designing the diffuse reflective cavity for higher initial irradiance and/or for longer retention time of the air in the cavity to increase the UV dose to the level required for the desired contaminant reduction level.

Although the invention has been described with reference to the above examples, it will be understood that modifications and variations are encompassed within the spirit and scope of the invention. Accordingly, the invention is limited only by the following claims.

What is claimed is:

1. An air treatment system, comprising:
a chamber, the chamber comprising a plurality of input apertures and a plurality of output apertures, wherein the interior surfaces of the chamber are substantially covered by a diffuse reflective material that is greater than 95% reflective to ultraviolet (UV) light; and
at least one continuous, narrow-band UV light source disposed within the chamber, wherein the light source is configured to emit greater than 90% of its output energy at wavelengths between 240 nm and 280 nm.

2. The air treatment system of claim 1, wherein the at least one continuous, narrow-band UV light source comprises a low-pressure mercury discharge lamp.

3. The air treatment system of claim 1, wherein at least a portion of the light source extends through an aperture in a wall or end plate of the chamber, and a portion of the light source disposed outside of the chamber provides at least one of mechanical securement or electrical connection for the light source.

4. The air treatment system of claim 1, wherein a ratio of the sum of the open area and interior surface area uncovered by the diffuse reflective material to the total surface area of the interior of the chamber walls is less than 0.05.

5. The air treatment system of claim 4, wherein the ratio of the sum of the open area and interior surface area uncovered by the diffuse reflective material to the total surface area of the interior of the chamber walls is less than 0.01.

6. The air treatment system of claim 1, wherein the chamber comprises a input end plate including the plurality of input apertures and an output end plate including the plurality of output apertures, wherein a ratio of open area of each of the end plates to the total area of each of the end plates is less than 0.4.

7. The air treatment system of claim 6, wherein the ratio of open area of each of the end plates to the total area of each of the end plates is less than 0.1.

8. The air treatment system of claim 1, wherein the air treatment system is configured to provide an irradiance of at least 75,000 μW/cm² at any location within the chamber.

9. The air treatment system of claim 8, wherein the air treatment system is configured to provide an irradiance of at least 100,000 μW/cm² at any location within the chamber.

10. The air treatment system of claim 9, wherein the air treatment system is configured to provide an irradiance of at least 150,000 μW/cm² at any location within the chamber.

11. The air treatment system of claim 1, wherein the air treatment system is configured to provide a UV dose of at least 150,000 μW-s/cm² to air flowing through the chamber with a dwell time within the chamber of less than 2 seconds.

12. The air treatment system of claim 1, wherein the air treatment system is configured to provide a UV dose of at least 150,000 μW-s/cm² to air flowing through the chamber with a dwell time within the chamber of less than 1 second.

13. An air treatment system, comprising:
a chamber, the chamber comprising a plurality of input apertures and a plurality of output apertures, wherein the interior surfaces of the chamber are substantially covered by a diffuse reflective material that is greater than 95% reflective to ultraviolet (UV) light; and
at least one continuous UV light source disposed within the chamber, wherein the air treatment system is configured to increase the irradiance at any location within the chamber to at least 10 times the irradiance of the at least one UV light source.

14. The air treatment system of claim 13, wherein the air treatment system is configured to provide an irradiance of at least 75,000 μW/cm² at any location within the chamber.

15. The air treatment system of claim 13, wherein the at least one UV light source comprises a narrow-band UV light source.

16. The air treatment system of claim 13, wherein at least a portion of the light source extends through an aperture in a wall or end plate of the chamber, and a portion of the light source disposed outside of the chamber provides at least one of mechanical securement or electrical connection for the light source.

17. An air treatment system for reducing an amount of a contaminant in air passing through the treatment system, the system comprising:
a chamber, the chamber comprising a plurality of input apertures and a plurality of output apertures to allow passage of air containing a contaminant therethrough, wherein the amount of contaminant in the air can be reduced by exposure to ultraviolet (UV) light, and wherein the interior surfaces of the chamber are substantially covered by a diffuse reflective material that is greater than 95% reflective to UV light; and
at least one UV light source disposed within the chamber, wherein the air treatment system is configured to increase the irradiance at any location within the chamber by a factor of at least 10.

18. The air treatment system of claim 17, wherein the at least one UV light source comprises a narrow-band UV light source.

19. The air treatment system of claim 17, wherein at least a portion of the light source extends through an aperture in a wall or end plate of the chamber, and a portion of the light source disposed outside of the chamber provides one of mechanical securement or electrical connection for the light source.

20. The air treatment system of claim 17, wherein the air treatment system is configured to provide an irradiance of at least 75,000 μW/cm² at any location within the chamber.

21. The air treatment system of claim 17, wherein the air treatment system is configured to reduce the amount of ozone in air passing through the chamber.

22. A method of reducing the presence of a contaminant in air, the method comprising:
directing air containing a contaminant into a chamber, wherein the amount of the contaminant can be reduced by exposure to ultraviolet (UV) light, the chamber having interior surfaces which are substantially covered by a diffuse reflective material that is greater than 95% reflective to UV light; and
introducing UV light into the chamber during passage of the air containing ozone therethrough, wherein the UV light is reflected multiple times by the interior surfaces of the chamber to provide an irradiance of at least 75,000 μW/cm² at any location within the chamber.

23. The method of claim 22, wherein the contaminant comprises ozone.

24. The method of claim 22, wherein introducing UV light into the chamber comprises exposing the air to an irradiance of at least 75,000 μW/cm² at any location within the chamber.
